SEQUENCE LISTING (1 sequence)

SEQ ID NO: 1 - DNA, 1427 bp, Burkholderia latens
cctccttgcggttagactagccacttctggtaaaacccactcccatggtgtgacgggcggtgtgtacaagacccgggaacgtattcaccgcggcatgctgatccgcgattactagcgattccagcttcatgcactcgagttgcagagtgcaatccggactacgatcggttttctgggattagctccccctcgcgggttggcaaccctctgttccgaccattgtatgacgtgtgaagccctacccataagggccatgaggacttgacgtcatccccaccttcctccggtttgtcaccggcagtctccttagagtgctcttgcgtagcaactaaggacaagggttgcgctcgttgcgggacttaacccaacatctcacgacacgagctgacgacagccatgcagcacctgtgcgccggttctctttcgagcactcccgaatctcttcaggattccgaccatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttactaaggaaatgaatccccaacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcctgccagtcaccaatgcagttcccaggttgagcccggggatttcacatcggtcttagcaaaccgcctgcgcacgctttacgcccagtaattccgattaacgcttgcaccctacgtattaccgcggctgctggcacgtagttagccggtgcttattcttccggtaccgtcatcccccggctgtattagagccaaggatttctttccggacaaaagtgctttacaacccgaaggccttcttcacacacgcggcattgctggatcaggctttcgcccattgtccaaaattccccactgctgcctcccgtaggagtctgggccgtgtctcagtcccagtgtggctggtcgtcctctcagaccagctactgatcgtcgccttggtaggcctttaccccaccaactagctaatcagccatcggccaaccctatagcgcgaggcccgaaggtcccccgctttcatccgtagatcgtatgcggtattaatccggctttcgccgggctatcccccactacaggacatgttccgatgtattactcacccgttcgccactcgccaccaggtgcaagcacccgtgctgccgttcgacttgcatgtgtaaggcatgccgccagcgttcaatctgag

(19) United States
(12) Patent Application Publication
(10) Pub. No.: US 2022/0256860 A1
(43) Pub. Date: Aug. 18, 2022

(54) BIOLOGICAL PREPARATION AND APPLICATION THEREOF

(71) Applicant: SHANDONG PEANUT RESEARCH INSTITUTE, Qingdao (CN)

(72) Inventors: Jie SUN, Qingdao (CN); Mingqing WANG, Qingdao (CN); Chushu ZHANG, Qingdao (CN); Lina YU, Qingdao (CN); Jie BI, Qingdao (CN); Yu SONG, Qingdao (CN); Jiancheng ZHANG, Qingdao (CN); Qingli YANG, Qingdao (CN)

(21) Appl. No.: 17/190,821

(22) Filed: Mar. 3, 2021

(30) Foreign Application Priority Data
Jan. 29, 2021 (CN) 202110126914.2
Jan. 29, 2021 (CN) 202110129515.1
Jan. 29, 2021 (CN) 202110129538.2

(51) Int. Cl. A01N 63/20; A01N 25/04; C12R 1/01; C12N 1/20

(52) U.S. Cl. CPC A01N 63/20; C12N 1/20; C12R 1/01; A01N 25/04

(57) ABSTRACT

Disclosed in the present invention is a biological preparation and application thereof, relates to the technical field of microorganisms, which includes a bacterial suspension or an anaculture or a crude extract or an extracellular metabolite of Burkholderia latens, wherein the Burkholderia latens is Burkholderia latens G12, preserved in CGMCC (No. 20817), the present invention has a good inhibition effect on Fusarium graminearum and Aspergillus flavus, and the infection with the Fusarium graminearum or Aspergillus flavus on the grains of the wheat or peanut in a storage period can be obviously reduced, and the storage period of the wheat or peanut can be prolonged. In addition, the present invention has a good degradation effect on aflatoxin.

### FIELD OF THE INVENTION

The present invention relates to the technical field of microorganisms, and particularly relates to a biological preparation and an application thereof.

### BACKGROUND

In recent years, with the rapid development of ecological agriculture, people have increasingly higher demand for the quality of agricultural products; green, safe and organic agricultural products gradually become more popular than fresh and high-quality products; and chemical pesticides are replaced with novel biological pesticides step by step. In addition, the use of the chemical pesticide in a traditional agricultural planting mode not only causes reduction of beneficial bacterial flora in soil, soil hardening, water pollution and reduction in crop yield, but also poses a threat to ecological diversity and ecosystem stability. Therefore, the development of a novel biological pesticide is significant for the transformation from traditional agriculture to ecological agriculture.

Fusarium graminearum is one of main pathogenic bacteria causing wheat scab which resulted in serious cereal yield reduction and lower grain quality. In addition, wheat grains might be infected with the Fusarium graminearum in the storage process of wheat since the grain itself carries germs or the storage condition is improper. After infecting the wheat, the Fusarium graminearum will produce a variety of toxic mycotoxins, including deoxynivalenol, zearalenone and nivalenol. Humans and animals will suffer from weakened immunity, deformity, cancer, or serious health problems after eating wheat with excessive toxins. Therefore, wheat grains infected with the Fusarium graminearum shall no longer be ground, consumed, or used as feed.

Aspergillus flavus belongs to Deuteromycetes and is a common saprophytic fungus, which is common in grain and oil food such as moldy peanuts, corn and feed. Some strains of the Aspergillus flavus can produce aflatoxins, which might cause toxic hepatitis, liver cirrhosis, liver cancer, or even death, seriously endangering the health of humans and animals. The aflatoxin is a group of compounds with similar chemical structures and exists in soil, animals, plants, and various nuts, especially peanuts and walnuts. The hazards of aflatoxin to human and animal health are related to the inhibition of protein synthesis by aflatoxin.

### BRIEF SUMMARY

One objective of the present invention is to provide a biological preparation, which includes a bacterial suspension or an anaculture or a crude extract or an extracellular metabolite of Burkholderia latens, wherein the Burkholderia latens is Burkholderia latens G12; and the strain is preserved in China General Microbiological Culture Collection Center (CGMCC) on Sep. 25, 2020, with the preservation number being CGMCC No. 20817, and the preservation address being 3, No.1 Beichen West Road, Chaoyang District, Beijing, China.

In some embodiments, a concentration of the Burkholderia latens G12 in the bacterial suspension or anaculture of the Burkholderia latens of the biological preparation is greater than or equal to 2.8×10⁸ cfu/mL.

In some embodiments, the anaculture of the Burkholderia latens is prepared through the following steps: inoculating an LB liquid culture medium with a cryopreserved bacterial liquid of the Burkholderia latens G12, to activate a strain; and then inoculating a new LB liquid culture medium with an activated bacterial liquid of the Burkholderia latens G12, shaking and culturing same for 36-48 h at 37° C. until a concentration of the bacterial liquid is greater than or equal to 2.8×10⁸ cfu/mL, so as to obtain the anaculture.

In some embodiments, the bacterial suspension of the Burkholderia latens G12 is a bacterial suspension with a concentration greater than or equal to 2.8 x 10⁸ cfu/mL prepared by centrifugating the anaculture of the Burkholderia latens G12, then discarding a supernatant, washing a wet thallus obtained through separation with sterile water, and finally adding sterile water to resuspend the thallus.

In some embodiments, the extracellular metabolite of Burkholderia latens G12 is prepared by filtering the supernatant, obtained after centrifugating the anaculture of the Burkholderia latens G12, through a filter membrane, and removing the thallus.

The second objective of the present invention is to provide a method for preventing Fusarium graminearum and Aspergillus flavus from infecting stored wheat and peanuts. In several specific embodiments, a bacterial suspension or extracellular metabolite of Burkholderia latens is specifically used for being sprayed on grains of the wheat or peanut; after spraying, the grain is air-dried in a shady area, placed at a room temperature and stored in a ventilative and dried mode; and wherein the Burkholderia latens is Burkholderia latens G12 with the preservation number of CGMCC No. 20817.

In some embodiments, when the bacterial suspension or extracellular metabolite of the Burkholderia latens is sprayed on the grains of the wheat or peanut, 2 mL of bacterial suspension or extracellular metabolite of the Burkholderia latens is sprayed on per 20 g of the peanuts or wheat.

In some embodiments, when the bacterial suspension of the Burkholderia latens is sprayed on the grains of the wheat or peanut, a concentration of the Burkholderia latens G12 in the bacterial suspension of the Burkholderia latens is greater than or equal to 2.8×10⁸ cfu/mL.

In some embodiments, the bacterial suspension of the Burkholderia latens G12 used when sprayed on the grains of the wheat or peanut is the bacterial suspension with a concentration greater than or equal to 2.8×10⁸ cfu/mL prepared by centrifugating an anaculture of the Burkholderia latens G12, then discarding a supernatant, washing a wet thallus obtained through separation with sterile water, and finally adding sterile water to resuspend the thallus.

In some embodiments, the extracellular metabolite of Burkholderia latens G12 used when sprayed on the grains of the wheat or peanut is prepared by filtering the supernatant, obtained after centrifugating the anaculture of the Burkholderia latens G12, through a filter membrane, and removing the thallus.

In some embodiments, the anaculture of the Burkholderia latens used to prepare the bacterial suspension or extracellular metabolite of the Burkholderia latens for being sprayed on the grains of the wheat or peanut is prepared through the following steps: inoculating an LB liquid culture medium with a cryopreserved bacterial liquid of the Burkholderia latens G12, to activate a strain; and then inoculating a new LB liquid culture medium with the activated bacterial liquid of the Burkholderia latens G12, shaking and culturing same for 36-48 h at 37° C. until a concentration of the bacterial liquid is greater than or equal to 2.8×10⁸ cfu/mL, so as to obtain the anaculture.

The third objective of the present invention is to provide a method for degrading aflatoxins. In several specific embodiments, the method comprising the following steps: adding an anaculture of Burkholderia latens into a to-be-degraded sample, then placing the sample at 30° C. and incubating for 3 days in the dark; and wherein the Burkholderia latens is Burkholderia latens G12 with the preservation number of CGMCC No. 20817.

In one specific embodiment, when the anaculture of the Burkholderia latens is used to degrade the aflatoxin, a bacterial concentration of the used anaculture of the Burkholderia latens is greater than or equal to 2.8×10⁸ cfu/mL.

In one specific embodiment, the anaculture of the Burkholderia latens for degrading the aflatoxin is prepared through the following steps: inoculating an LB liquid culture medium with a cryopreserved bacterial liquid of the Burkholderia latens G12, to activate a strain; and then inoculating a new LB liquid culture medium with the activated bacterial liquid of the Burkholderia latens G12, shaking and culturing same for 36-48 h at 37° C. until a concentration of the bacterial liquid is greater than or equal to 2.8×10⁸ cfu/mL, so as to obtain the anaculture.

In the above-mentioned solutions, the used to-be-degraded sample is cereal, feed or food contaminated with the aflatoxin, wherein in one specific embodiment, the sample is peanut meal contaminated with the aflatoxin.

In one specific embodiment, when the anaculture of the Burkholderia latens is used to degrade the aflatoxin, 10 mL of anaculture of the Burkholderia latens is added to per 20 g of the to-be-degraded samples.

The advantages of the technical solutions of the present invention lie in that the present invention obtains one Burkholderia latens strain by separating same from seawater, the biological preparation prepared by the Burkholderia latens has a good inhibition effect on Fusarium graminearum and Aspergillus flavus, the grains of the wheat or peanut are treated with the bacterial suspension or the anaculture or a crude extract or the extracellular metabolite of the Burkholderia latens G12 of the present invention, and thus the infection with the Fusarium graminearum or Aspergillus flavus on the grains of the wheat or peanut in a storage period can be obviously reduced, and the storage period of the wheat or peanut can be prolonged.

In addition, the biological preparation of the present invention has a good degradation effect on the aflatoxin, wherein a degradation rate of an aflatoxin B₁ is as high as 93.6%, and a degradation rate of an aflatoxin G₁ is as high as 73.3%; and when the biological preparation is used to detoxify the peanut meal contaminated with the aflatoxin, a result shows that a removal rate of the aflatoxin in the peanut meal is 89%, and it can be seen that the biological preparation has a good application prospect in the field of aflatoxin degradation and removal.

### BRIEF DESCRIPTION OF THE DRAWINGS

FIG. 1 is a diagram of colonial morphology of a strain G12;

FIG. 2 is an electrophoretogram of 16S rRNA of the strain G12 (1 denotes an amplified band of the 16S rRNA of the strain G12, and 2 denotes Markers);

FIG. 3 shows plate confrontation results of antagonizing Fusarium graminearum by the strain G12 (A: experimental group, the Fusarium graminearum is in the middle, and the periphery is inoculated with the strain G12; B: control group);

FIG. 4 shows results of preventing the Fusarium graminearum from infecting stored wheat by a biological preparation 1 (Fusarium graminearum spores are added into wheat in a bottle A, and Fusarium graminearum spores and the biological preparation 1 are added into wheat in a bottle B);

FIG. 5 shows plate confrontation results of antagonizing Aspergillus flavus by the strain G12;

FIG. 6 shows results of preventing the Aspergillus flavus from infecting stored peanuts by the biological preparation 1;

FIG. 7 is liquid chromatograms of an aflatoxin B₁ before and after degradation;

FIG. 8 is liquid chromatograms of an aflatoxin G₁ before and after degradation; and

FIG. 9 shows results of degrading an aflatoxin in a peanut meal sample by a biological preparation 5.

### DETAILED DESCRIPTION OF THE DISCLOSURE

The terms used in the present invention, unless otherwise indicated, generally have the meanings commonly understood by those of ordinary skill in the art.

The present invention is described in further detail below with reference to the specific embodiments and data. The following embodiments are merely to illustrate the present invention and are not intended to limit the scope of the present invention in any way.

### Embodiment 1: Separation, Purification and Identification of a Strain G12

(1) Separation and Purification

Bacteria were separated from seawater of a coastal area in Qingdao in May 2018 by using a dilution culture method, including the following steps:

Adding 1 mL of seawater into 100 mL of sterilized water for dilution; separating bacterial strains from a diluent on an LB solid plate; preliminarily classifying the bacterial strains according to characteristics of color, morphology, etc.; obtaining a pure bacterial strain numbered as G12 through streaking purification; and culturing the pure bacterial strain in an LB liquid culture medium, adding glycerol with a final concentration of 20%, and cryopreserving same in a refrigerator at −20° C. for standby.

(2) Identification of the Strain

According to a method described in “Berger's bacterial identification manual” (8th Edition), morphological characteristics and physiological and biochemical characteristics of the strain G12 are identified, and specific results are as follows:

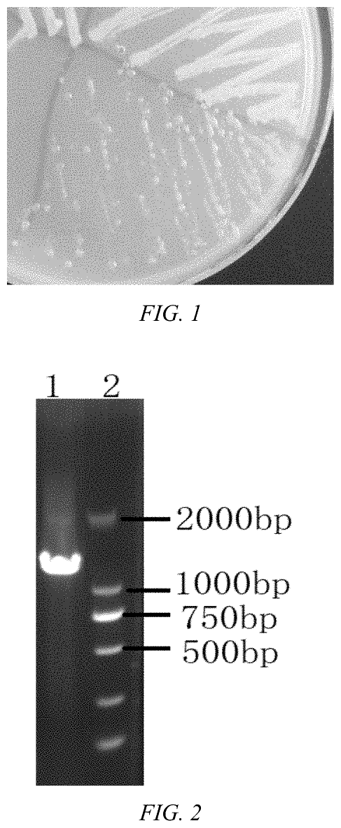

1. Morphological characteristics: a single colony of the strain G12 is raised and opaque on the LB culture medium, and the colony is about 5-8 mm after 2-day culture (FIG. 1).

2. The strain G12 has the following biological properties: the strain G12 can grow at 4-42° C., is Gram-negative, can utilize glucose, arabinose, D-mannose, D-mannitol and adipic acid, and cannot utilize maltose.

3. Gene analysis of 16S rRNA:

Extracting bacterial genome DNA of the G12, and performing PCR amplification by utilizing a 16S rRNA gene universal primer, wherein an agarose gel electrophoresis result is as shown in FIG. 2; and connecting an amplification product to a pMD19-T vector, transforming a recombinant plasmid into Escherichia coli, and sequencing to obtain a gene sequence. A gene of the 16S rRNA of the strain G12 has 100% homology with a gene of a 16S rRNA of a standard strain Burkholderia latens LMG 24064^{T} according to the sequence homology comparison between standard strains in the EzTaxon-e server database, and the gene analysis shows that the strain G12 is Burkholderia latens. The sequence of the 16S rRNA of the G12 is as shown in SEQ ID NO: 1.

The strain G12 is identified as the Burkholderia latens by combining the morphological characteristics, physiological and biochemical identification, 16S rDNA sequencing and homology analysis, named as Burkholderia latens G12, and is preserved in China General Microbiological Culture Collection Center (CGMCC) on Sep. 25, 2020 with the address: 3, No.1 Beichen West Road, Chaoyang District, Beijing, China, Institute of Microbiology, Chinese Academy of Sciences, and the postcode: 100101; and the preservation unit is Shandong Peanut Research Institute, and the preservation number is CGMCC No. 20817.

### Embodiment 2: Antagonistic Effect on Fusarium graminearum by a Strain G12

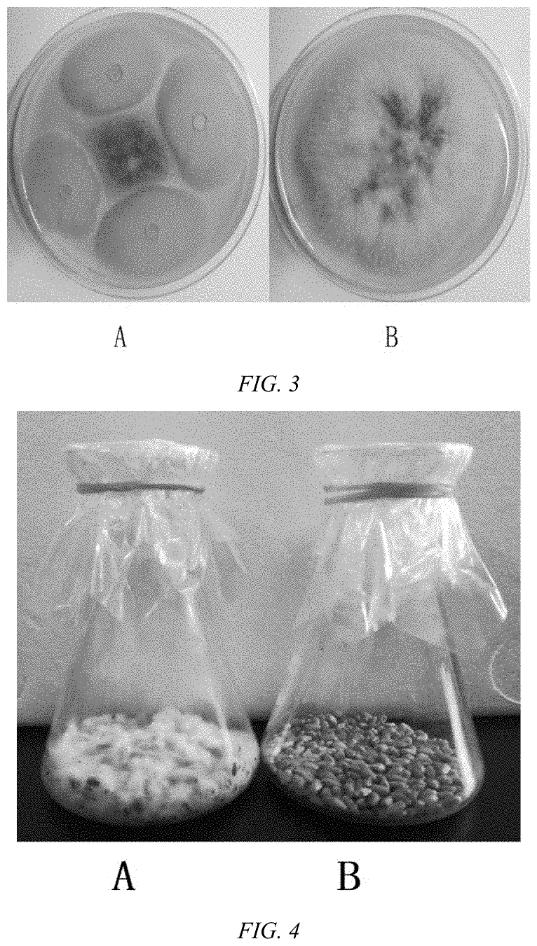

A plate confrontation culture method is used, including inoculating a PDA plate center with 10 μL of spore liquid (a concentration: 7.9×10⁵/mL) of Fusarium graminearum S7 (provided by the Gong Kuijie's research team of Crop Research Institute, Shandong Academy of Agricultural Sciences), and inoculating the periphery of the Fusarium graminearum with the purified strain G12 (20 μL, a concentration: 2.8×10⁸ cfu/mL) for antibacterial test, as an experimental group; taking a plate not inoculated with the strain G12, as a control group; and performing static culture for 7 days in an incubator at 28° C. It is found that the strain G12 can obviously inhibit the Fusarium graminearum from growing (FIG. 3) and is an effective bacterium antagonizing the Fusarium graminearum.

### Embodiment 3: Antagonizing Fusarium graminearum in Wheat by a Biological Preparation 1

Embodiment 3 includes: preparing the biological preparation 1: inoculating a test tube filled with 10 mL of LB liquid culture medium with 100 μL of cryopreserved bacterial liquid of Burkholderia latens G12, culturing same for 12 h to activate a strain, inoculating 100 mL of LB liquid culture medium with 500 μL of activated bacterial liquid, and shaking and culturing same for 36-48 h at 37° C. until a concentration of the bacterial liquid is 2.8×10⁸ cfu/mL, so as to obtain an anaculture of the Burkholderia latens G12;

Wherein the LB liquid fermentation culture medium (g/L) includes 10 g of tryptone, 5 g of yeast extracts and 10 g of NaCl and a pH is 7.0;

Putting 15 g of the wheat into each of two triangular bottles numbered as A and B;

Adding 2 mL of sterilized LB liquid culture medium into 10 μL spore suspension with a concentration of 7.9×10⁵/mL of Fusarium graminearum S7, fully mixing same and then adding the same into the bottle A, as a control group; and adding 2 mL of the biological preparation 1 into 10 μL of spore suspension with a concentration of 7.9×10⁵/mL of Fusarium graminearum S7, fully mixing same and then adding the same to the bottle B, to serve as an experimental group. After placement at 28° C. and culture for 7 days, as shown in FIG. 4, a surface of the wheat in the bottle A is overgrown with the Fusarium graminearum, and there is no Fusarium graminearum on a surface of the wheat in the bottle B, which shows that the biological preparation 1 can obviously inhibit the Fusarium graminearum in the wheat from growing.

### Embodiment 4: Preventing Fusarium graminearum from Infecting Stored Wheat by a Biological Preparation 2

Embodiment 4 includes: preparing the biological preparation 2: taking 10 mL of anaculture of the Burkholderia latens G12 prepared in Embodiment 3, wherein the concentration of the bacterial liquid is 2.8×10⁸ cfu/mL, centrifugating the anaculture at 8000 r/min for 10 min, so as to obtain a thallus and a supernatant through separation, washing the prepared thallus with sterile water and then centrifugating same, adding sterile water to make up to 10 mL, and suspending the thallus to obtain a bacterial suspension of the Burkholderia latens G12;

Dividing 200 g of the newly harvested wheat with full grains and uncontaminated with the Fusarium graminearum into two groups randomly, with every 100 g as one group, and arranging three parallels for each group; spraying a sterilized LB culture medium on a surface of wheat in the first group, wherein the spraying amount of 2 mL/20g, which is recorded as a control group; spraying the above-mentioned biological preparation 2 (the bacterial suspension with the bacterial concentration of 2.8×10⁸ cfu/mL of the Burkholderia latens G12) on a surface of wheat in the second group, wherein the spraying amount of 2 mL/20 g, which is recorded as an experimental group; placing the above-mentioned wheat in each group at a room temperature to be stored in a ventilative and dried mode after air-drying in a shady area; and sampling to detect the content of the Fusarium graminearum in the wheat every 30 days for a total of 90 days, wherein the content situation of the Fusarium graminearum in each group is as shown in Table 1.

TABLE 1
Contents of Fusarium graminearum (cfu/g)

| Number of days | 30 d | 60 d | 90 d |
|---|---|---|---|
| Control group | 1.2 × 10² | 6.1 × 10³ | 4.7 × 10⁶ |
| Experimental group | negative | 67 | 1.9 × 10² |

As seen from Table 1, with the increase of time, the content of the Fusarium graminearum in the experimental group is far less than that in the control group, which means that spraying the biological preparation 2 can effectively prevent contamination with the Fusarium graminearum in the wheat and prolong a storage period of the wheat.

### Embodiment 5: Preventing Fusarium graminearum from Infecting Stored Wheat by a Biological Preparation 3

Embodiment 5 includes: preparing the biological preparation 3: inoculating an LB liquid culture medium with a cryopreserved bacterial liquid of Burkholderia latens G12, culturing same for 12 h to activate a strain, then inoculating a new LB liquid culture medium with the activated bacterial liquid of the Burkholderia latens G12, and shaking and culturing same for 36-48 h at 37° C. until a concentration of the bacterial liquid is 2.8×10⁸ cfu/mL; centrifugating the bacterial liquid at 8000 rpm for 10 min, and then filtering same with a 0.22 μm filter membrane to remove a thallus, so as to prepare an extracellular metabolite of the Burkholderia latens G12;

Dividing 200 g of the newly harvested wheat with full grains and uncontaminated with the Fusarium graminearum into two groups randomly, with every 100 g as one group, and arranging three parallels for each group; spraying a sterilized LB culture medium on a surface of wheat in the first group, wherein the spraying amount of 2 mL/20 g, which is recorded as a control group; spraying the above-mentioned prepared biological preparation 3 on a surface of wheat in the second group, wherein the spraying amount of 2 mL/20 g, which is recorded as an experimental group; placing the above-mentioned wheat in each group at a room temperature to be stored in a ventilative and dried mode after air-drying in a shady area; and sampling to detect the content of the Fusarium graminearum in the wheat every 30 days for a total of 90 days, wherein the content situation of the Fusarium graminearum in each group is as shown in Table 2.

TABLE 2
Contents of Fusarium graminearum (cfu/g)

| Number of days | 30 d | 60 d | 90 d |
|---|---|---|---|
| Control group | 1.1 × 10² | 6.3 × 10³ | 6.2 × 10⁶ |
| Experimental group | negative | 97 | 2.3 × 10² |

As seen from Table 2, with the increase of time, the content of the Fusarium graminearum in the experimental group is far less than that in the control group, which means that spraying the biological preparation 3 can effectively prevent contamination with the Fusarium graminearum in the wheat and prolong a storage period of the wheat.

### Embodiment 6: Inhibition Effect on Aspergillus flavus by a Strain G12

A plate confrontation culture method is used to detect an inhibition effect on Aspergillus flavus by the strain G12, the method particularly including:

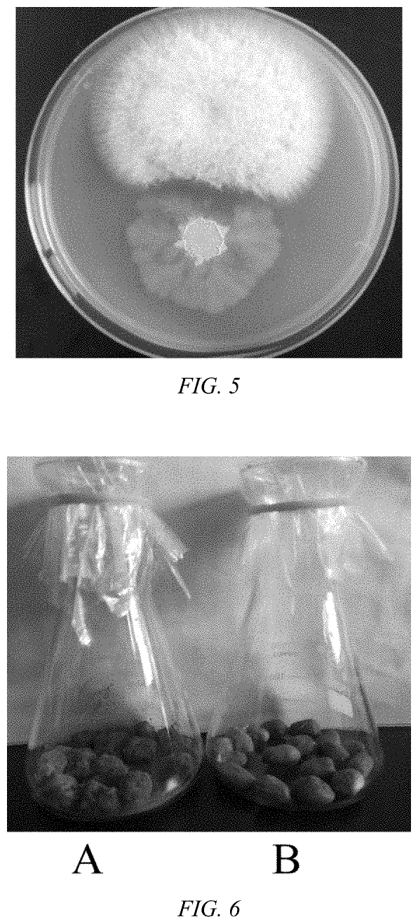

Inoculating a GY culture medium solid plate (a GY solid culture medium: 20 g of glucose, 5 g of yeast powder, 20 g of agar and 1000 mL of water) with 10 μL of spore liquid (a concentration of the spore liquid: 5.6×10⁵/mL) of an Aspergillus flavus NRRL 3357 standard strain kindly provided by Professor He Zhumei of Sun Yat-sen University, inoculating the periphery of the Aspergillus flavus with 50 μL of bacterial liquid (a concentration: 2.8×10⁸ cfu/mL) of the Burkholderia latens G12; and performing an antibacterial test, specifically, performing static culture for 7 days in an incubator at 28° C. A result is as shown in FIG. 5: the Aspergillus flavus is on an upper portion of the plate, the Burkholderia latens G12 is on a lower portion of the plate, and an Aspergillus flavus colony near the Burkholderia latens G12 is obviously inhibited.

### Embodiment 7: Antagonizing Aspergillus flavus in Peanuts by a Biological Preparation 1

Embodiment 7 includes: numbering bottles as A and B, and putting 18 peanuts in each bottle; adding 3 mL of sterilized LB culture medium into 10 μL of Aspergillus flavus spore with a concentration of 6.1×10⁵/mL, fully mixing same and then adding the same into the bottle A, which is recorded as a control group; and adding 3 mL of biological preparation 1 (an anaculture of a strain G12: the strain G12 is cultured in an LB liquid culture medium, and a concentration is 2.8×10⁸ cfu/mL) into 10 μL of Aspergillus flavus spore with a concentration of 5.6×10⁵/mL, fully mixing same and then adding the same into the bottle B. After placement at 28° C. and culture for 6 days, as shown in FIG. 6, a surface of the peanut in the bottle A is overgrown with the Aspergillus flavus which presents yellow green and almost covers the entire peanut, there is no Aspergillus flavus on a surface of the peanut in the bottle B, the surface of the peanut is clean and bright and presents normal color, which shows that the biological preparation 1 can obviously inhibit the Aspergillus flavus in the peanut from growing.

### Embodiment 8:

### Preventing Aspergillus flavus from Infecting Stored Peanuts by a Biological Preparation 2

Embodiment 8 includes: dividing 160 g of the newly harvested peanuts with full grains and uncontaminated with the Aspergillus flavus into two groups randomly, with every 80 g as one group, and arranging three parallels for each group; spraying a sterilized LB culture medium on a surface of a peanut in the first group, wherein the spraying amount of 2 mL/20g, which is recorded as a control group; spraying the biological preparation 2 (a bacterial suspension with a bacterial concentration of 2.8×10⁸ cfu/mL of Burkholderia latens G12) prepared in Embodiment 2 on a surface of a peanut in the second group, wherein the spraying amount of 2 mL/20g, which is recorded as an experimental group; placing the above-mentioned peanut in each group at a room temperature to be stored in a ventilative and dried mode after air-drying in a shady area; and sampling to detect the content of the Aspergillus flavus in the peanut every 30 days for a total of 90 days, wherein the content situation of the Aspergillus flavus in each group is as shown in Table 3.

TABLE 3
Contents of Aspergillus flavus (cfu/g)

| Number of days | 30 d | 60 d | 90 d |
|---|---|---|---|
| Control group | 2.1 × 10² | 9.2 × 10⁴ | 6.9 × 10⁶ |
| Experimental group | negative | 56 | 1.7 × 10² |

As seen from Table 3, with the increase of time, the content of the Aspergillus flavus in the experimental group is far less than that in the control group, which shows that spraying the biological preparation 2 can effectively prevent contamination with the Aspergillus flavus in the peanut and prolong a storage period of the peanut.

### Embodiment 9: Preventing Aspergillus flavus from Infecting Stored Peanuts by a Biological Preparation 3

Embodiment 9 includes: dividing 160 g of the newly harvested peanuts with full grains and uncontaminated with the Aspergillus flavus into two groups randomly, with every 80g as one group, and arranging three parallels for each group; spraying a sterilized LB culture medium on a surface of a peanut in the first group, wherein the spraying amount of 2 mL/20g, which is recorded as a control group; spraying the above-mentioned biological preparation 3 prepared in Embodiment 5 on a surface of a peanut in the second group, wherein the spraying amount of 2 mL/20g, which is recorded as an experimental group; placing the above-mentioned peanut in each group at a room temperature to be stored in a ventilative and dried mode after air-drying in a shady area; and sampling to detect the content of the Aspergillus flavus in the peanut every 30 days for a total of 90 days, wherein the content situation of the Aspergillus flavus in each group is as shown in Table 4.

TABLE 4
Contents of Fusarium graminearum (cfu/g)

| Number of days | 30 d | 60 d | 90 d |
|---|---|---|---|
| Control group | 2.2 × 10² | 9.4 × 10⁴ | 6.8 × 10⁶ |
| Experimental group | negative | 59 | 1.8 × 10² |

As seen from Table 4, with the increase of time, the content of the Aspergillus flavus in the experimental group is far less than that in the control group, which shows that spraying the biological preparation 3 can effectively prevent contamination with the Aspergillus flavus in the peanut and prolong a storage period of the peanut.

### Embodiment 10: Degradation Effect on an Aflatoxin B₁ by a Biological Preparation 4

Embodiment 10 includes: preparing the biological preparation 4: inoculating a test tube filled with 10 mL of LB liquid culture medium with 100 μL of cryopreserved bacterial liquid of Burkholderia latens G12, culturing same for 12 h to activate a strain, inoculating 100 mL of LB liquid culture medium with 500 μL of activated bacterial liquid, and shaking and culturing same for 36-48 h at 37° C. until a concentration of the bacterial liquid is 2.9×10⁸ cfu/mL, so as to obtain an anaculture of the Burkholderia latens G12;

Adding 1980 μL of biological preparation 4 into 20 μL of 10 mg/kg aflatoxin B₁ standard, and incubating for 3 days at 30° C. in the dark;

Centrifugating the incubated liquid at 10000 r/min for 10 min, filtering a supernatant with a 0.22 μm filter membrane and then making the supernatant pass through an immunoaffinity column, washing twice with ultrapure water, then eluting with chromatographic-grade methanol, and collecting eluant; and detecting the content of the aflatoxin B₁ in a solution by using high performance liquid chromatography, wherein the detection conditions of the high performance liquid chromatography (HPLC) are as follows: a C-18 chromatographic column (4.6 mm×15 cm×5 μm), a sample size: 20 μL, a mobile phase: methanol:water=1:1 (V/V), a flow rate: 0.8 mL/min, an excitation wavelength of a fluorescence detector: 360 nm and an emission wavelength: 440 nm. A degradation rate of the aflatoxin B₁ is calculated by utilizing the following formula:

$$Y = ( 1 - \frac{S}{C} ) \times 100 \%$$

C denotes a peak area of the aflatoxin B₁ before degradation, S denotes a peak area of a residual aflatoxin B₁ in a sample treated with the biological preparation 4, and Y denotes the degradation rate of the aflatoxin B₁.

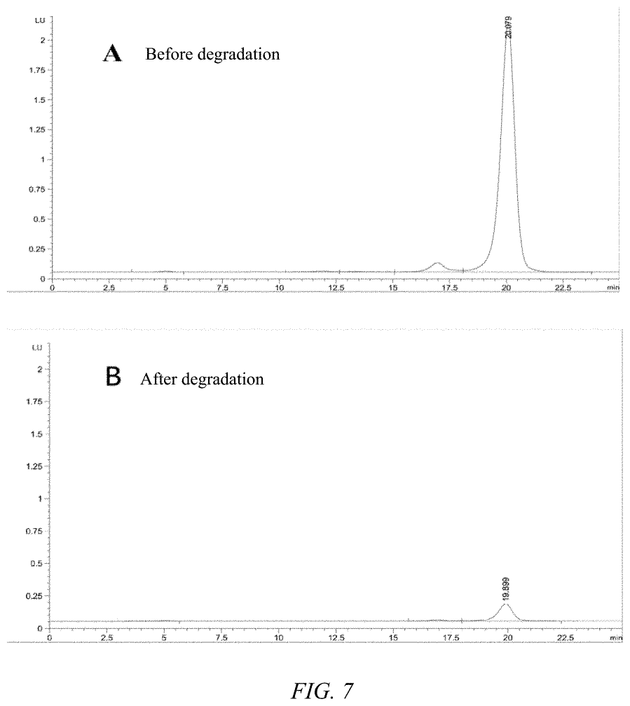

A detection result is as shown in FIG. 7, after incubation with the biological preparation 4, the degradation rate of the aflatoxin B₁ is as high as 93.6%, and the degradation rate is high.

### Embodiment 11: Degradation Effect on an Aflatoxin G₁ by a Biological Preparation 4

Embodiment 11 includes: adding 1980 μL of biological preparation 4 prepared in Embodiment 10 into 10 μL of 5 mg/kg AFG₁ standard, and incubating for 3 days at 30° C. in the dark;

Centrifugating the incubated liquid at 10000 r/min for 10 min, filtering a supernatant with a 0.22 μm filter membrane and then making the supernatant pass through an immunoaffinity column, washing twice with ultrapure water, then eluting with chromatographic-grade methanol, and collecting eluant; and detecting the content of the aflatoxin G₁ in a solution by using high performance liquid chromatography, wherein the detection conditions of the high performance liquid chromatography (HPLC) are as follows: a C-18 chromatographic column (4.6 mm×15 cm×5 μm), a sample size: 20 μL, a mobile phase: methanol:water=1:1 (V/V), a flow rate: 0.8 mL/min, an excitation wavelength of a fluorescence detector: 360 nm and an emission wavelength: 440 nm. A degradation rate of the aflatoxin G₁ is calculated by utilizing the following formula:

$$Y = ( 1 - \frac{S}{C} ) \times 100 \%$$

C denotes a peak area of the aflatoxin G₁ before degradation, S denotes a peak area of a residual aflatoxin G₁ in a sample treated with the biological preparation 4, and Y denotes the degradation rate of the aflatoxin G₁.

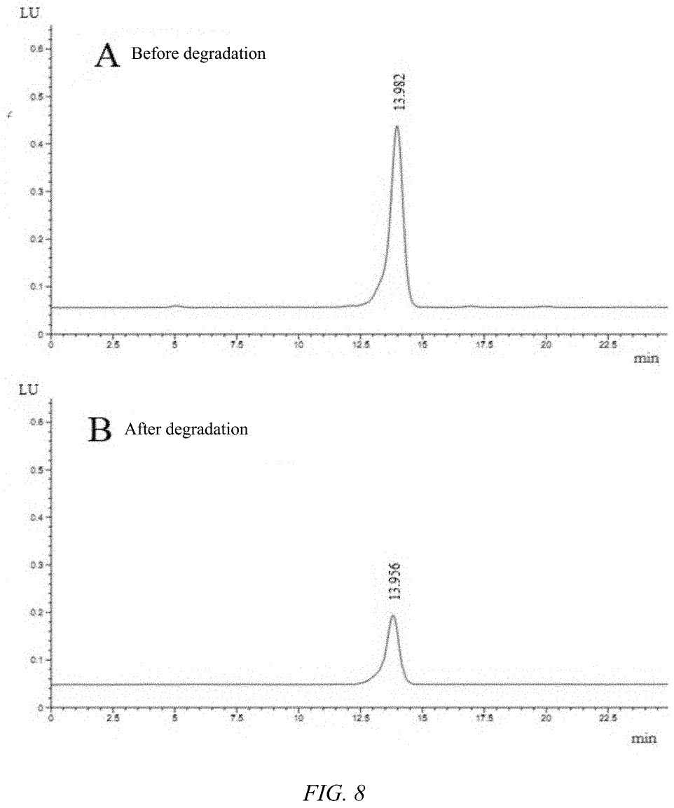

A detection result is as shown in FIG. 8, after incubation with a strain G12, the aflatoxin G₁ is degraded, the degradation rate is as high as 73.3%, and the result shows that the biological preparation 4 can degrade the aflatoxin G₁.

### Embodiment 12: Degradation Effect on an Aflatoxin in a Peanut Sample by a Biological Preparation 5

Embodiment 12 includes: preparing the biological preparation 5: inoculating a test tube filled with 10 mL of LB liquid culture medium with 100 μL of cryopreserved bacterial liquid of Burkholderia latens G12, culturing same for 12 h to activate a strain, inoculating 100 mL of LB liquid culture medium with 500 μL of activated bacterial liquid, and shaking and culturing same for 36-48 h at 37° C. until a concentration of the bacterial liquid is 4.7×10⁸ cfu/mL, so as to obtain an anaculture of the Burkholderia latens G12;

Taking peanut meal samples with aflatoxins B₁ exceeding a standard as research objects, and detecting the degradation condition of the aflatoxin in peanut meal by the biological preparation 5, which includes the following steps:

Taking 20 g of peanut meal samples with an aflatoxin B₁ exceeding the standard and added with 10 mL of sterilized fermentation culture medium after sterilization as a sample S1, serving as a control group; and taking 20 g of peanut meal samples with an aflatoxin B₁ exceeding the standard and added with 10 mL of biological preparation 5 (the anaculture with a concentration of 4.7×10⁸ cfu/mL of a strain G12) after sterilization as a sample S2, serving as an experimental group.

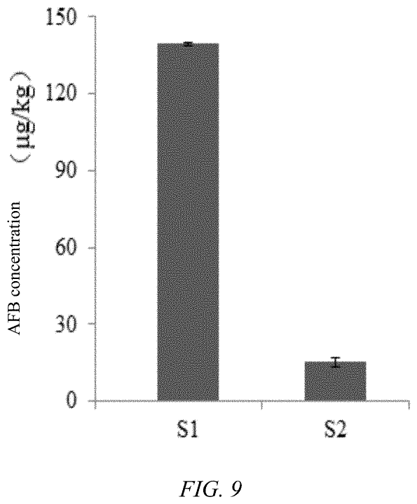

After the two groups are incubated for 72 h at 30° C., detection results of the contents of the aflatoxins in the two samples are as shown in FIG. 9: the content of the aflatoxin B₁ in the sample S1 is 139 μg/kg, while the content of the aflatoxin B₁ in the sample S2, which is treated by being added with the biological preparation 5 and incubated for 72 h, is significantly reduced to 15 μg/kg. By means of treatment with the biological preparation 5, the aflatoxin B₁ in the peanut meal can be obviously reduced by 89%. Therefore, the biological preparation 5 of the present invention can obviously degrade the aflatoxin B₁ in the peanut sample with the aflatoxin exceeding the standard.

1. A biological preparation, comprising a bacterial suspension or an anaculture or a crude extract or an extracellular metabolite of Burkholderia latens, wherein the Burkholderia latens is Burkholderia latens G12; and the strain is preserved in China General Microbiological Culture Collection Center (CGMCC) on Sep. 25, 2020, with the preservation number being CGMCC No. 20817, and the preservation address being 3, No.1 Beichen West Road, Chaoyang District, Beijing, China.

2. The biological preparation according to claim 1, a concentration of the Burkholderia latens G12 in the bacterial suspension or anaculture of the Burkholderia latens of the biological preparation is greater than or equal to 2.8×10⁸ cfu/mL.

3. The biological preparation according to claim 1, the anaculture of the Burkholderia latens is prepared through the following steps: inoculating an LB liquid culture medium with a cryopreserved bacterial liquid of the Burkholderia latens G12, to activate a strain; and then inoculating a new LB liquid culture medium with an activated bacterial liquid of the Burkholderia latens G12, shaking and culturing same for 36-48 h at 37° C. until a concentration of the bacterial liquid is greater than or equal to 2.8×10⁸ cfu/mL, so as to obtain the anaculture.

4. The biological preparation according to claim 1, the bacterial suspension of the Burkholderia latens G12 is a bacterial suspension with a concentration greater than or equal to 2.8×10⁸ cfu/mL prepared by centrifugating the anaculture of the Burkholderia latens G12, then discarding a supernatant, washing a wet thallus obtained through separation with sterile water, and finally adding sterile water to resuspend the thallus.

5. The biological preparation according to claim 1, the extracellular metabolite of Burkholderia latens G12 is prepared by filtering the supernatant, obtained after centrifugating the anaculture of the Burkholderia latens G12, through a filter membrane, and removing the thallus.

6. A method for preventing Fusarium graminearum or Aspergillus flavus from infecting stored wheat and peanuts, a bacterial suspension or extracellular metabolite of Burkholderia latens is specifically used for being sprayed on grains of the wheat or peanut; after spraying, the grain is air-dried in a shady area, placed at a room temperature and stored in a ventilative and dried mode; and wherein the Burkholderia latens is Burkholderia latens G12 with the preservation number of CGMCC No. 20817.

7. The method for preventing Fusarium graminearum or Aspergillus flavus from infecting stored wheat and peanuts according to claim 6, 2 mL of bacterial suspension or extracellular metabolite of the Burkholderia latens is sprayed on per 20 g of the peanuts or wheat.

8. The method for preventing Fusarium graminearum or Aspergillus flavus from infecting stored wheat and peanuts according to claim 6, a concentration of the Burkholderia latens G12 in the bacterial suspension of the Burkholderia latens is greater than or equal to 2.8×10⁸ cfu/mL.

9. The method for preventing Fusarium graminearum or Aspergillus flavus from infecting stored wheat and peanuts according to claim 8, the bacterial suspension of the Burkholderia latens G12 used when sprayed on the grains of the wheat or peanut is the bacterial suspension with a concentration greater than or equal to 2.8×10⁸cfu/mL prepared by centrifugating an anaculture of the Burkholderia latens G12, then discarding a supernatant, washing a wet thallus obtained through separation with sterile water, and finally adding sterile water to resuspend the thallus.

10. The method for preventing Fusarium graminearum or Aspergillus flavus from infecting stored wheat and peanuts according to claim 6, the extracellular metabolite of Burkholderia latens G12 used when sprayed on the grains of the wheat or peanut is prepared by filtering the supernatant, obtained after centrifugating the anaculture of the Burkholderia latens G12, through a filter membrane, and removing the thallus.

11. The method for preventing Fusarium graminearum or Aspergillus flavus from infecting stored wheat and peanuts according to claim 9, the anaculture of the Burkholderia latens is prepared through the following method: inoculating an LB liquid culture medium with a cryopreserved bacterial liquid of the Burkholderia latens G12, to activate a strain; and then inoculating a new LB liquid culture medium with the activated bacterial liquid of the Burkholderia latens G12, shaking and culturing same for 36-48 h at 37° C. until a concentration of the bacterial liquid is greater than or equal to 2.8×10⁸ cfu/mL, so as to obtain the anaculture.

12. A method for degrading aflatoxins, the method comprising the following steps: adding an anaculture of Burkholderia latens into a to-be-degraded sample, then placing the sample at 30° C. and incubating for 3 days in the dark; and wherein the Burkholderia latens is Burkholderia latens G12 with the preservation number of CGMCC No. 20817.

13. The method for degrading aflatoxins according to claim 12, a bacterial concentration of the used anaculture of the Burkholderia latens is greater than or equal to 2.8×10⁸ cfu/mL.

14. The method for degrading aflatoxins according to claim 13, the anaculture of the Burkholderia latens is prepared through the following steps: inoculating an LB liquid culture medium with a cryopreserved bacterial liquid of the Burkholderia latens G12, to activate a strain; and then inoculating a new LB liquid culture medium with the activated bacterial liquid of the Burkholderia latens G12, shaking and culturing same for 36-48 h at 37° C. until a concentration of the bacterial liquid is greater than or equal to 2.8×10⁸ cfu/mL, so as to obtain the anaculture.

15. The method for degrading aflatoxins according to claim 12, the used to-be-degraded sample is cereal, feed or food contaminated with the aflatoxin.

16. The method for degrading aflatoxins according to claim 12, 10 mL of anaculture of the Burkholderia latens is added to per 20 g of the to-be-degraded samples.
